(19) United States
(12) Patent Application Publication
(10) Pub. No.: US 2025/0031703 A1
(43) Pub. Date: Jan. 30, 2025

(54) USE OF PECTIN OR PECTIN-RELATED SACCHARIDES TO ENHANCE EFFICACY OF PLANT GROWTH-PROMOTING RHIZOBACTERIA (PGPR) STRAINS FOR PROMOTING GROWTH AND HEALTH IN PLANTS AND ANIMALS

(71) Applicant: AUBURN UNIVERSITY, Auburn, AL (US)

(72) Inventors: Mark R. Liles, Auburn, AL (US); Joseph Kloepper, Auburn, AL (US)

(73) Assignee: AUBURN UNIVERSITY, Auburn, AL (US)

(21) Appl. No.: 18/797,756

(22) Filed: Aug. 8, 2024

Related U.S. Application Data
(63) Continuation of application No. 17/553,350, filed on Dec. 16, 2021, now Pat. No. 12,089,598.
Application No. 17/553,350 is a continuation of application No. 16/560,620, filed on Sep. 4, 2019, now abandoned.
Application No. 16/560,620 is a continuation of application No. 15/473,975, filed on Mar. 30, 2017, now Pat. No. 10,888,593.
Application No. 15/473,975 is a continuation-in-part of application No. PCT/US2015/053239, filed on Sep. 30, 2015.
(60) Provisional application No. 62/057,667, filed on Sep. 30, 2014.

(51) Int. Cl. A01N 63/22; A01H 3/00; A23K 10/18; A23K 20/163; A23K 50/80; A61K 31/7004; A61K 31/732; A61K 35/74; A61K 35/742; A61K 48/00; A61P 1/00

(52) U.S. Cl. CPC A01N 63/22; A01H 3/00; A23K 10/18; A23K 20/163; A23K 50/80; A61K 31/7004; A61K 31/732; A61K 35/74; A61K 35/742; A61K 48/00; A61P 1/00

(57) ABSTRACT

To understand the growth-promoting and disease-inhibiting activities of plant growth-promoting rhizobacteria (PGPR) strains, the genomes of 12 Bacillus subtilis group strains with PGPR activity were sequenced and analyzed. These B. subtilis strains exhibited high genomic diversity, whereas the genomes of B. amyloliquefaciens strains (a member of the B. subtilis group) are highly conserved. A pairwise BLASTp matrix revealed that gene family similarity among Bacillus genomes ranges from 32-90%, with 2,839 genes within the core genome of B. amyloliquefaciens subsp. plantarum (now B. velezensis). Comparative genomic analyses of B. amyloliquefaciens strains identified genes that are linked with biological control and colonization of roots and/or leaves, including 73 genes uniquely associated with subsp. plantarum (now B. velezensis) strains that have predicted functions related to signaling, transportation, secondary metabolite production, and carbon source utilization. Although B. amyloliquefaciens (now B. velezensis) strains contain gene clusters that encode many different secondary metabolites, only polyketide biosynthetic clusters that encode difficidin and macrolactin are conserved within this subspecies. To evaluate their role in plant pathogen biocontrol, genes involved in secondary metabolite biosynthesis were deleted in B. amyloliquefaciens (now B. velezensis) strain, revealing that difficidin expression is critical in reducing the severity of disease, caused by Xanthomonas axonopodis pv. vesicatoria in tomato plants. This Example defines genomic features of PGPR strains and links them with biocontrol activity and with host colonization.

### CROSS-REFERENCE TO RELATED APPLICATIONS

The present application is a continuation under 35 U.S.C. § 120 of U.S. application Ser. No. 17/553,350, filed Dec. 16, 2021, which is a continuation U.S. patent application Ser. No. 16/560,620, filed on Sep. 4, 2019, now abandoned, which is a continuation under 35 U.S.C. § 120, of U.S. application Ser. No. 15/473,975, filed on Mar. 30, 2017, and issued as U.S. Pat. No. 10,888,593, on Jan. 12, 2021, which is a continuation-in-part (CIP) under 35 U.S.C. § 365 (c) of International Application No. PCT/US2015/053239, filed on Sep. 30, 2015, which international application claims the benefit of priority under 35 U.S.C. § 119 (c) to U.S. provisional application No. 62/057,667, filed on Sep. 30, 2014, the content of which applications are incorporated herein by reference in their entireties.

### SEQUENCE LISTING

A Sequence Listing accompanies this application and is submitted as an ASCII text file of the sequence listing named “2021-12-16_169996-00473_Seq_Listing” which is 55.0 kilo bytes in size and was created on Dec. 16, 2021. The sequence listing is electronically submitted via EFS-Web with the application and is incorporated herein by reference in its entirety.

### FIELD

The presently disclosed subject matter relates to the field of plant growth-promoting rhizobacteria (PGPR). In particular, the present subject matter relates to the use of pectin or pectin-related saccharide to enhance the efficacy of PGPR in regard to promoting growth and health in plants and animals.

### BACKGROUND

Plant-associated microorganisms have been extensively examined for their roles in natural and induced suppressiveness of soilborne diseases. Among the many groups of such organisms are root-associated bacteria, which generally represent a subset of soil bacteria. Rhizobacteria are a subset of total rhizosphere bacteria which have the capacity, upon re-introduction to seeds or vegetative plant parts (such as potato seed pieces), to colonize the developing root system in the presence of competing soil microflora. Root colonization is typically examined by quantifying bacterial populations on root surfaces; however, some rhizobacteria can also enter roots and establish at least a limited endophytic phase. Hence, root colonization may be viewed as a continuum from the rhizosphere to the rhizoplane to internal tissues of roots.

Rhizobacteria which exert a beneficial effect on the plant being colonized are termed “plant-growth promoting rhizobacteria” or “PGPR.” PGPR may benefit the host by causing plant growth promotion or biological disease control. The same strain of PGPR may cause both growth promotion and biological control. Among the soilborne pathogens shown to be negatively affected by PGPR are Aphanomyces spp., Fusarium oxysporum, Gaeumannomyces graminis, Phytophthora spp., Pythium spp., Rhizoctonia solani, Sclerotium rolfsii, Thielaviopsis basicola, and Verticillium spp. In most of these cases, biological control results from bacterial production of metabolites which directly inhibit the pathogen, such as antibiotics, hydrogen cyanide, iron-chelating siderophores, and cell wall-degrading enzymes. Plant growth promotion by PGPR may also be an indirect mechanism of biological control, leading to a reduction in the probability of a plant contracting a disease when the growth promotion results in shortening the time that a plant is in a susceptible state, e.g. in the case where PGPR cause enhanced seedling emergence rate, thereby reducing the susceptible time for pre-emergence damping-off. An alternative mechanism for biological control by PGPR is induced systemic resistance. PGPR and uses thereof are disclosed in the prior art. (See, e.g., U.S. Pat. Nos. 8,445,255; 6,524,998; 5,935,839; 5,640,803; 5,503,652; and 5,503,651; the contents of which are incorporated herein by reference in their entirety).

In addition to their observed association in nature with plants, PGPR also may be utilized as probiotics for animals in order to improve animal growth or animal health. For example, Bacillus amyloliquefaciens subsp. plantarum (now Bacillus velezensis) AP193 has been described as a probiotic for fish. (See U.S. Published Application No. 2012/0328572).

In swine, probiotics have been used to have a positive influence on gut microbiota balance, intestinal epithelium integrity and maturation of gut-associated tissue. (See Corcionivoshi et al., Animal Science and Biotechnologies, 2010, 43 (1)). In poultry, probiotics have been used to maintain digestive microbial balance and to reduce potential pathogenic bacteria which results in improving growth, egg production, and feed conversion. (See id.). In cattle, probiotics have been used to prevent and combat digestive disorders such as diarrhea during lactation, to influence ruminal metabolism of nutrients, which helps maintain health and improve productive performance. (See id.). In sheep, probiotics have been used to prevent and combat pathological conditions that arise from digestive balance. (See id.).

Therefore, new compositions and methods of use for PGPR in promoting growth and health in plants and animals are desirable.

### SUMMARY

Disclosed are compositions and methods that include or utilize plant growth promoting rhizobacteria (PGPR) for improving growth and health in plants and animals. The compositions and methods include or utilize a plant growth promoting rhizobacteria (PGPR) that expresses a protein associated with pectin metabolism, and a saccharide comprising pectin or a pectin-related saccharide.

The disclosed compositions may include inoculants which comprise: (a) a plant growth promoting rhizobacteria (PGPR) that expresses a protein associated with pectin metabolism; and (b) a saccharide comprising pectin or a pectin-related saccharide. Suitable PGPR may include Bacillus species such as Bacillus amyloliquefaciens subspecies plantarum (now Bacillus velezensis). The pectin or pectin-related saccharides may include pectin-derived saccharides such as hydrolyzed pectin, D-galacturonate, D-glucuronate, or mixtures thereof. Optionally, the pectin or pectin-related saccharide functions as a carrier for the PGPR and/or the inoculant includes a carrier other than the pectin or pectin-related saccharide.

The disclosed compositions may be used to treat plants, seeds, and soils in order to improve plant growth or plant health. The disclosed compositions may be formulated as a plant treatment composition, a coating for seeds, or a soil amendment composition.

The disclosed compositions also may be administered to animals in order to improve animal growth or animal health. The disclosed compositions may be formulated as an animal feed, such as a pelleted animal feed.

Also disclosed are methods of using pectin or pectin-related saccharides and PGPR in regard to promoting growth or health in plants and animals. The disclosed methods for improving plant growth or plant health may include: (a) treating plants, seeds, or soil with a plant growth promoting rhizobacteria (PGPR) that expresses a protein associated with pectin metabolism and (b) treating the plants, seeds, or soil with a saccharide comprising pectin or a pectin-related saccharide, where the plants, seeds, or soil may be treated with the PGPR and the saccharide concurrently or are treated with the PGPR and saccharide non-currently in either order.

The disclosed methods for improving animal growth or animal health may include (a) administering to an animal a plant growth promoting rhizobacteria (PGPR) that expresses a protein associated with pectin metabolism and (b) administering to the animal a saccharide comprising pectin or a pectin-related saccharide, where the animals may be administered the PGPR and the saccharide concurrently or are treated with the PGPR and saccharide non-currently in either order.

Also disclosed are methods of using pectin or pectin-related saccharides and PGPR in regard to promoting and/or increasing nodulation in nitrogen-fixing plants such as nitrogen-fixing legumes. The disclosed methods for promoting and/or increasing nodulation in a legume may include: (a) treating the legume, seeds of the legume, or soil surrounding the legume with a plant growth promoting rhizobacteria (PGPR) that expresses a protein associated with pectin metabolism and (b) treating the legume, seeds of the legume, or soil surrounding the legume with a saccharide comprising pectin or a pectin-related saccharide, where the legume, seeds of the legume, or soil surrounding the legume may be treated with the PGPR and the saccharide concurrently or the legume, seeds of the legume, or soil surrounding the legume are treated with the PGPR and saccharide non-currently in either order.

Also disclosed are methods of using pectin or pectin-related saccharides to prepare compositions and inoculants as disclosed herein. The methods may include combining PGPR and pectin, which has been extracted from pectin-containing plant material, or pectin-related saccharides to prepare the disclosed compositions and inoculants. Optionally, a carrier may be combined with the PGPR and pectin or pectin-related saccharides to prepare the disclosed compositions and inoculants.

### BRIEF DESCRIPTION OF THE FIGURES

FIG. 1. Expression of a pectin lyase activity by PGPR Bap (now Bv) strain AP193. Note the cleared halo around the growth of the Bap (now Bv) strain due to pectin degradation.

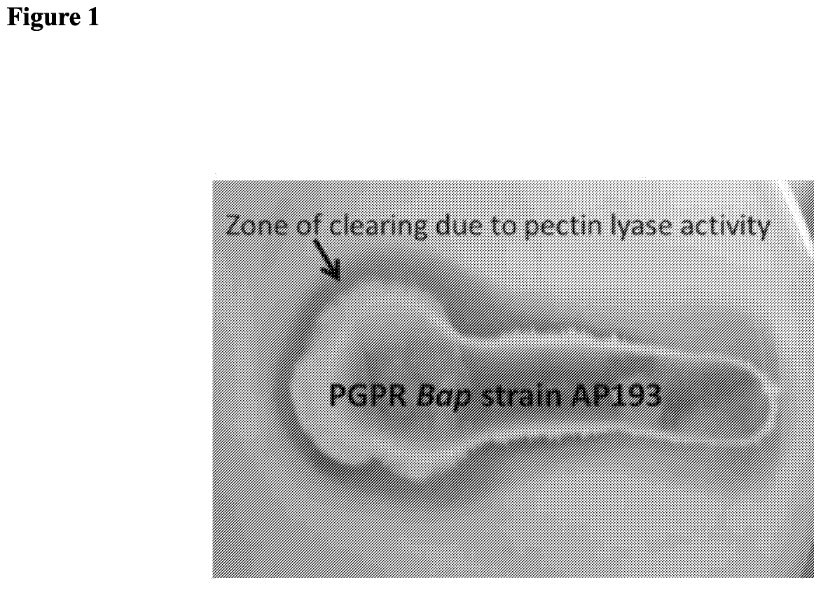

FIG. 2. Use of 1% pectin as a sole C source by PGPR strains AP143 and AP193 in TSS medium. The small increase in OD₆₀₀ by the non-PGPR strain HD73 was due to residual nutrients present from the previous TSB culture.

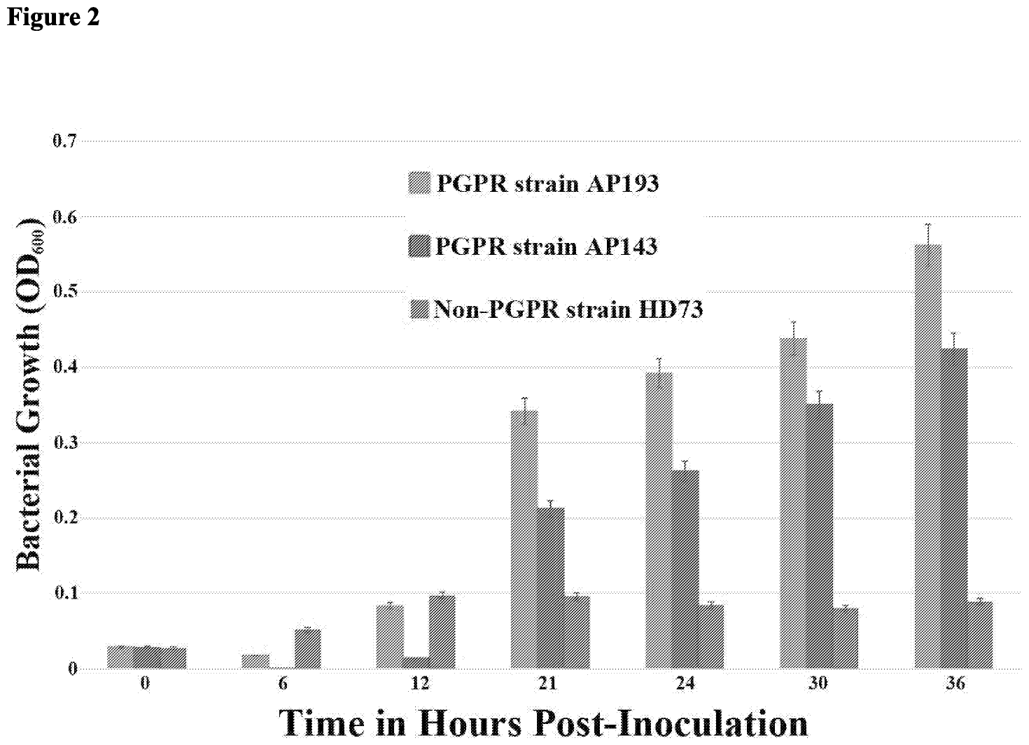

FIG. 3. Effect of PGPR and/or 0.1% (w/w) pectin soil amendment on soybean root and growth after 4 weeks. Bv strains AP143 and AP193 were applied as seed inoculants with 10⁶ CFU/seed and plants (n=11/treatment) were grown in a greenhouse. Treatment groups assigned different letters are significantly different (P<0.5).

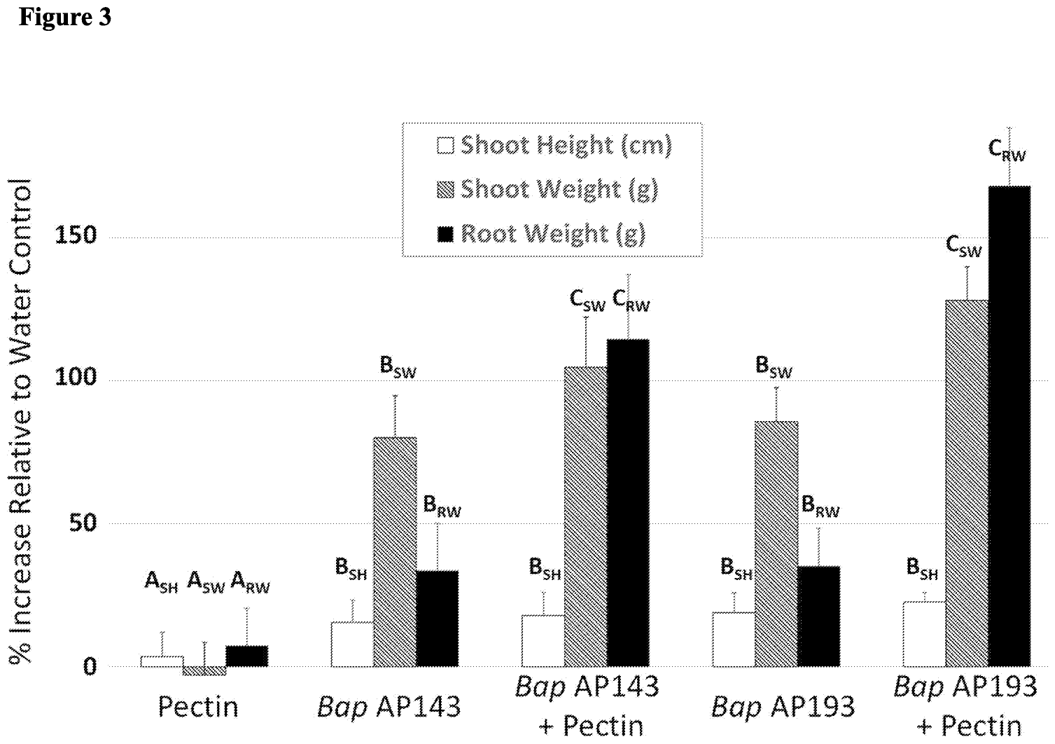

FIG. 4. A. Representative soybean roots grown in the presence of absence of 0.1% pectin amended to soil and seed inoculated with 10⁶ CFU Bv strain AP142. B. Effect of treatment groups that include 10⁶ CFU/seed Bv strains, with and without 0.1% pectin, on soybean nodulation (n=11). Treatment groups assigned different letters are significantly different (P<0.5).

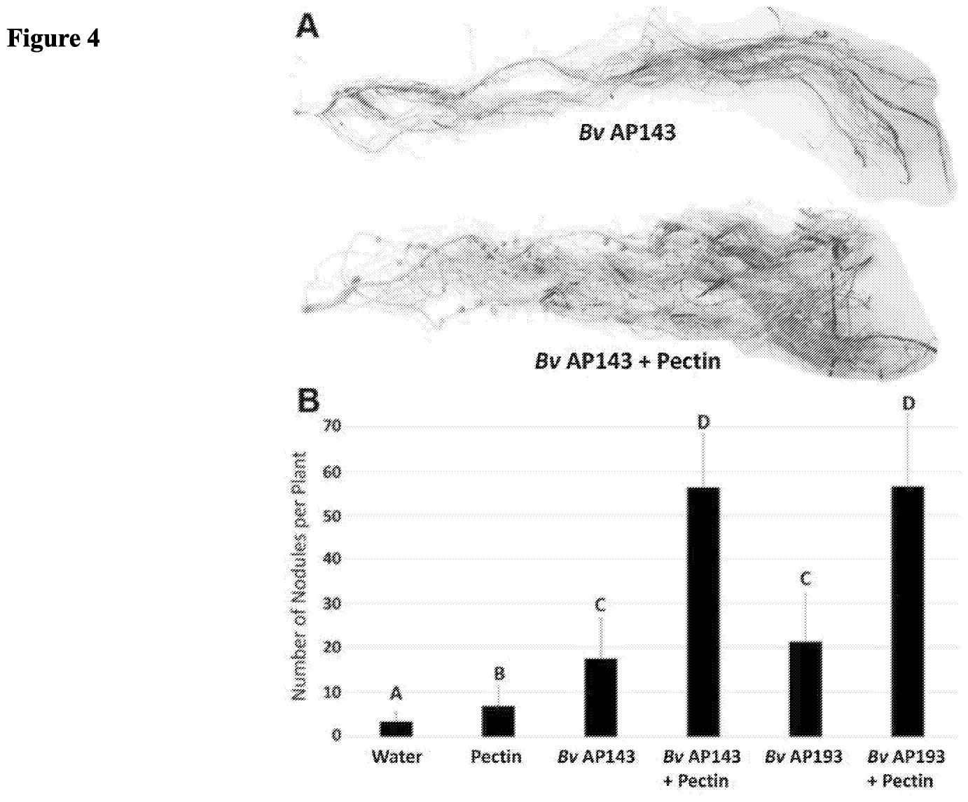

### DETAILED DESCRIPTION

The disclosed subject matter of the invention may be described using various terms as described below.

Unless otherwise specified or indicated by context, the terms “a”, “an”, and “the” mean “one or more.” For example, “a sugar” should be interpreted to mean “one or more sugars” unless otherwise specified or indicated by context.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The term “plant” as utilized herein should be interpreted broadly and may include angiosperms and gymnosperms, dicots and monocots, and trees. Examples of angiosperm dicots may include, but are not limited to tomato, tobacco, cotton, rapeseed, field beans, soybeans, peppers, lettuce, peas, alfalfa, clover, cabbage, broccoli, cauliflower, brussel sprouts), radish, carrot, beets, eggplant, spinach, cucumber, squash, melons, cantaloupe, and sunflowers. Example of angiosperm monocots may include, but are not limited to asparagus, field and sweet com, barley, wheat, rice, sorghum, onion, pearl millet, rye, oats, and sugar cane. Woody plants may include, but are not limited to fruit trees, acacia, alder, aspen, beech, birch, sweet gum, sycamore, poplar, willow, fir, pine, spruce, larch, cedar, and hemlock.

The term “plant” may include nitrogen-fixing plants such as nitrogen-fixing legumes. As is understood in the art, a “legume” is a plant belonging to the family Facaceae or Leguminosas. Most legumes have root nodules comprising symbiotic nitrogen-fixing bacteria. The symbiotic nitrogen-fixing bacteria of root nodules are capable of taking atmospheric nitrogen (N₂) and reducing the atmospheric N₂ to ammonia via the reaction: N₂+8H⁺+8e⁻→2NH₃+H₂. The ammonia thus produced can be further reduced to ammonium by the following reaction: NH₃+H⁺→NH₄⁺. The ammonium thus produced can be used by the legume as a nitrogen source for growth. As such, root nodules and the symbiotic nitrogen-fixing bacteria therewithin are important for plant growth and methods and compositions that promote and/or increase nodulation are desirable.

The term “animal” as utilized herein should be interpreted broadly and may include mammals and non-mammals. Mammals may include human and non-human mammals, such as cows, pigs, sheep, and the like. Non-mammals may include birds (e.g., chickens, turkeys, ducks, and the like) and fish.

Non-human animals may include aquatic animals. In particular, aquatic animals may include farmed fish (e.g. catfish or tilapia) and crustaceans (e.g., shrimp).

The present inventors have identified a collection of plant growth-promoting rhizobacteria (PGPR) that are capable of improving the growth of plants, and also have disease- and pest-controlling activity. From an analysis of genome sequences from the best-performing Bacillus amyloliquefaciens subspecies plantarum (now Bacillus velezensis) PGPR strains, the inventors identified some genetically encoded functions that are always present within these Bacillus PGPR strains and are not present in other Bacillus species that are not plant-related. In particular, these PGPR strains can use sugars derived from plant pectin as a carbon and/or energy source. By supplementing pectin on plant seeds that are inoculated with Bacillus spores, or by supplementing the amount of pectin available for Bacillus PGPR strain post-seed germination, this will result in an enhancement of 1) the Bacillus strain colonization of the plant rhizosphere and/or 2) better persistence of Bacillus within the plant rhizosphere and/or 3) better plant growth performance in response to PGPR strain+pectin administration and/or 4) better biological control of disease (e.g., bacteria, fungi, viruses) or pests (e.g., nematodes) as a result of PGPR strain+pectin administration.

### PGPR

The term “plant growth promoting rhizobacteria” or “PGPR” refers to a group of bacteria that colonize plant roots, and in doing so, promote plant growth and/or reduce disease or damage from predators. Bacteria that are PGPR may belong to genera including, but not limited to Actinobacter, Alcaligenes, Bacillus, Burkholderia, Buttiauxella, Enterobacter, Klebsiella, Kluyvera, Pseudomonas, Rahnella, Ralstonia, Rhizobium, Serratia, Stenotrophomonas, Paenibacillus, and Lysinibacillus. The PGPR utilized in the disclosed methods and composition may be a single strain, species, or genus of bacteria or may comprise a mixture of bacterial strains, species, or genera. For example, the PGPR may be selected from genera including, but not limited to, Actinobacter, Alcaligenes, Bacillus, Burkholderia, Buttiauxella, Enterobacter, Klebsiella, Kluyvera, Pseudomonas, Rahnella, Ralstonia, Rhizobium, Serratia, Stenotrophomonas, Paenibacillus, and Lysinibacillus.

The genus Bacillus as used herein refers to a genus of Gram-positive, rod-shaped bacteria which are members of the division Firmicutes. Under stressful environmental conditions, the Bacillus bacteria produce oval endospores that can stay dormant for extended periods. Bacillus bacteria may be characterized and identified based on the nucleotide sequence of their 16S rRNA or a fragment thereof (e.g., approximately a 1000 nt, 1100 nt, 1200 nt, 1300 nt, 1400 nt, or 1500 nt fragment of 16S rRNA or rDNA nucleotide sequence). Bacillus bacteria may include, but are not limited to B. acidiceler, B. acidicola, B. acidiproducens, B. aeolius, B. aerius, B. aerophilus, B. agaradhaerens, B. aidingensis, B. akibai, B. alcalophilus, B. algicola, B. alkalinitrilicus, B. alkalisediminis, B. alkalitelluris, B. altitudinis, B. alveayuensis, B. amyloliquefaciens, B. anthracis, B. aquimaris, B. arsenicus, B. aryabhattai, B. asahii, B. atrophaeus, B. aurantiacus, B. azotoformans, B. badius, B. barbaricus, B. bataviensis, B. beijingensis, B. benzoevorans, B. beveridgei, B. bogoriensis, B. boroniphilus, B. butanolivorans, B. canaveralius, B. carboniphilus, B. cecembensis, B. cellulosilyticus, B. cereus, B. chagannorensis, B. chungangensis, B. cibi, B. circulans, B. clarkii, B. clausii, B. coagulans, B. coahuilensis, B. cohnii, B. decisifrondis, B. decolorationis, B. drentensis, B. farraginis, B. fastidiosus, B. firmus, B. flexus, B. foraminis, B. fordii, B. fortis, B. fumarioli, B. funiculus, B. galactosidilyticus, B. galliciensis, B. gelatini, B. gibsonii, B. ginsengi, B. ginsengihumi, B. graminis, B. halmapalus, B. halochares, B. halodurans, B. hemicellulosilyticus, B. herbertsteinensis, B. horikoshi, B. horneckiae, B. horti, B. humi, B. hwajinpoensis, B. idriensis, B. indicus, B. infantis, B. infernus, B. isabeliae, B. isronensis, B. jeotgali, B. koreensis, B. korlensis, B. kribbensis, B. krulwichiae, B. lehensis, B. lentus, B. licheniformis, B. litoralis, B. locisalis, B. luciferensis, B. luteolus, B. macauensis, B. macyae, B. mannanilyticus, B. marisflavi, B. marmarensis, B. massiliensis, B. megaterium, B. methanolicus, B. methylotrophicus, B. mojavensis, B. muralis, B. murimartini, B. mycoides, B. nanhaiensis, B. nanhaiisediminis, B. nealsonii, B. neizhouensis, B. niabensis, B. niacini, B. novalis, B. oceanisediminis, B. odysseyi, B. okhensis, B. okuhidensis, B. oleronius, B. oshimensis, B. panaciterrae, B. patagoniensis, B. persepolensis, B. plakortidis, B. pocheonensis, B. polygoni, B. pseudoalcaliphilus, B. pseudofirmus, B. pseudomycoides, B. psychrosaccharolyticus, B. pumilus, B. qingdaonensis, B. rigui, B. ruris, B. safensis, B. salarius, B. saliphilus, B. schlegelii, B. selenatarsenatis, B. selenitireducens, B. seohaeanensis, B. shackletonii, B. siamensis, B. simplex, B. siralis, B. smithii, B. soli, B. solisalsi, B. sonorensis, B. sporothermodurans, B. stratosphericus, B. subterraneus, B. subtilis, B. taeansis, B. tequilensis, B. thermantarcticus, B. thermoamylovorans, B. thermocloacae, B. thermolactis, B. thioparans, B. thuringiensis, B. tripoxylicola, B. tusciae, B. vallismortis, B. vedderi, B. vietnamensis, B. vireti, B. wakoensis, B. weihenstephanensis, B. xiaoxiensis, and mixtures or blends thereof.

The PGPR and inoculants thereof disclosed herein may include B. amyloliquefaciens or a Bacillus species that is closely related to B. amyloliquefaciens. A Bacillus species that is closely related to B. amyloliquefaciens may be defined as a species having a 16S rDNA sequence comprising SEQ ID NO:26 or comprising a 16S rDNA sequence having at least about 98% or 99% sequence identity to SEQ ID NO:26. The PGPR preferably is B. amyloliquefaciens subspecies plantarum (now B. velezensis) or a Bacillus species that is closely related to B. amyloliquefaciens subspecies plantarum (now B. velezensis). B. amyloliquefaciens subspecies plantarum (now B. velezensis) is a subspecies of B. amyloliquefaciens which is colonizes plant roots and typically exhibits amylase activity. Suitable PGPR strains for the disclosed methods and compositions may include PGPR strains having a gyrB gene that exhibits sequence identity to the gyrB gene from strains of Bacillus amyloliquefaciens subspecies plantarum (now Bacillus velezensis). In some embodiment, the PGPR strain utilized in the disclosed methods and compositions has at gyrB gene having at least about 80%, 90%, 95%, 96%, 97%, 98%, 99% or 100% sequence identity to the polynucleotide sequence of SEQ ID NO:25, which is the polynucleotide sequence of the gyrB gene from strains of Bacillus amyloliquefaciens subsp. plantarum (now Bacillus velezensis).

Suitable strains of B. amlyoliquefaciens subsp. plantarum (B. velezensis) for use in the disclosed compositions and methods may include but are not limited to Bacillus amyloliquefaciens subsp. plantarum (now Bacillus velezensis) AS43.3, Bacillus amyloliquefaciens subsp. plantarum (now Bacillus velezensis) TrigoCor1448, Bacillus amyloliquefaciens subsp. plantarum (now Bacillus velezensis) UCMB5033, Bacillus amyloliquefaciens subsp. plantarum (now Bacillus velezensis) UCMB5113, Bacillus amyloliquefaciens subsp. plantarum (now Bacillus velezensis) EBL11, Bacillus amyloliquefaciens subsp. plantarum (now Bacillus velezensis) W2, Bacillus amyloliquefaciens subsp. plantarum (now Bacillus velezensis) UCMB5036, Bacillus amyloliquefaciens subsp. plantarum (now Bacillus velezensis) IT-45, Bacillus amyloliquefaciens subsp. plantarum (now Bacillus velezensis) UASWS BA1, Bacillus amyloliquefaciens subsp. plantarum (now Bacillus velezensis) LFB 112, Bacillus amyloliquefaciens subsp. plantarum (now Bacillus velezensis) CAUB946, Bacillus amyloliquefaciens subsp. plantarum (now Bacillus velezensis) M27, Bacillus amyloliquefaciens subsp. plantarum (now Bacillus velezensis) B1895, Bacillus amyloliquefaciens subsp. plantarum (now Bacillus velezensis) SQR9, Bacillus amyloliquefaciens subsp. plantarum (now Bacillus velezensis) AH159-1, Bacillus amyloliquefaciens subsp. plantarum (now Bacillus velezensis) DC-12, Bacillus amyloliquefaciens subsp. plantarum (now Bacillus velezensis) YAU B9601-Y2, Bacillus amyloliquefaciens subsp. plantarum (now Bacillus velezensis) Y2, Bacillus amyloliquefaciens subsp. plantarum (now Bacillus velezensis) EGD_AQ14, Bacillus amyloliquefaciens subsp. plantarum (now Bacillus velezensis) NAU-B3, Bacillus amyloliquefaciens subsp. plantarum (now Bacillus velezensis) FZB42, Bacillus amyloliquefaciens subsp. plantarum (now Bacillus velezensis) CC178, Bacillus amyloliquefaciens subsp. plantarum (now Bacillus velezensis) AP79, Bacillus amyloliquefaciens subsp. plantarum (now Bacillus velezensis) AP71, Bacillus amyloliquefaciens subsp. plantarum (now Bacillus velezensis) AP143, Bacillus amyloliquefaciens subsp. plantarum (now Bacillus velezensis) AP193, Bacillus amyloliquefaciens subsp. plantarum (now Bacillus velezensis) AB01, and Bacillus amyloliquefaciens subsp. plantarum (now Bacillus velezensis) GB03.

Suitable PGPR strains and inoculants thereof for the methods and compositions disclosed herein may include PGPR strains that express one or more proteins associated with pectin metabolism. In some embodiments, the PGPR strain may express one or more proteins associated with pectin metabolism, which may include but are not limited to proteins encoded by a gene selected from the group consisting of uxaA (altronate dehydratase), uxaB (altronate oxidoreductase), uxaC (uronate isomerase), uxaA (mannonate dehydratase, uxuB (D-mannonate oxidoreductase), kdgA (4-hydroxy-2-oxoglutarate aldolase), kdgK (2-dehydro-3-deoxygluconokinase), exuR (hexuronate utilization operon transcriptional repressor), exuT (hexuronate transporter), and combinations thereof. In some embodiments, the PGPR strain may express one or more pectinase enzymes selected from a group consisting of pectin lyase, pectate lyase, polygalacturonase, and pectin esterase.

The uxaA gene encodes an enzyme which is an altronate dehydratase (EC: 4.2.1.7) which converts D-altronate to 2-dehydro-3-deoxy-D-gluconate and water. Therefore, suitable PGPR strains and inoculants thereof for the methods and composition disclosed herein may include a PGPR strain that expresses altronate dehydratase. SEQ ID NO:1 provides the polynucleotide sequence encoding for altronate dehydratase. SEQ ID NO:2 provides the amino acid sequence for altronate dehydratase.

The uxaB gene encodes an enzyme which is an altronate oxidoreductase (EC: 5.3.1.12) which converts D-altronate and NAD⁺ to D-tagaturonate and NADH. Therefore, suitable PGPR strains and inoculants thereof for the methods and composition disclosed herein may include a PGPR strain that expresses altronate oxidoreductase. SEQ ID NO:3 provides the polynucleotide sequence encoding for altronate oxidoreductase. SEQ ID NO:4 provides the amino acid sequence for altronate oxidoreductase.

The uxaC gene encodes an enzyme which is an uronate isomerase (EC: 1.3.1.12) which converts D-glucuronate to D-fructuronate and which converts D-galacturonate to D-tagaturonate. Therefore, suitable PGPR strains and inoculants thereof for the methods and composition disclosed herein may include a PGPR strain that expresses uronate isomerase. SEQ ID NO:5 provides the polynucleotide sequence encoding for altronate oxidoreductase. SEQ ID NO:6 provides the amino acid sequence for altronate oxidoreductase.

The uxuA gene encodes an enzyme which is a mannonate dehydratase (EC: 4.2.1.8) which converts D-mannonate to 2-dehydro-3-deoxy-D-gluconate. Therefore, suitable PGPR strains and inoculants thereof for the methods and composition disclosed herein may include a PGPR strain that expresses mannonate dehydratase. SEQ ID NO:7 provides the polynucleotide sequence encoding for mannonate dehydratase. SEQ ID NO:8 provides the amino acid sequence for mannonate dehydratase.

The uxuB gene encodes an enzyme which is a D-mannonate oxidoreductase (EC: 1.1.1.57) which converts D-mannonate and NAD⁺ to D-fructuronate and NADH. Therefore, suitable PGPR strains and inoculants thereof for the methods and composition disclosed herein may include a PGPR strain that expresses D-mannonate oxidoreductase. SEQ ID NO: 9 provides the polynucleotide sequence encoding for altronate oxidoreductase. SEQ ID NO: 10 provides the amino acid sequence for altronate oxidoreductase.

The kdgA gene encodes an enzyme which is a 4-hydroxy-2-oxoglutarate aldolase (EC 4.1.3.16) which converts 4-hydroxy-2-oxoglutarate to pyruvate and glyoxylate, and which converts 2-dehydro-3-deoxy-6-phosphate-D-gluconate to pyruvate and D-glyceraldehyde 3-phosphate. Therefore, suitable PGPR strains and inoculants thereof for the methods and composition disclosed herein may include a PGPR strain that expresses 4-hydroxy-2-oxoglutarate aldolase. SEQ ID NO:11 provides the polynucleotide sequence encoding for 4-hydroxy-2-oxoglutarate aldolase. SEQ ID NO:12 provides the amino acid sequence for 4-hydroxy-2-oxoglutarate aldolase.

The kdgK gene encodes an enzyme which is 2-dehydro-3-deoxygluconokinase (EC 2.7.1.45) which phosphorylates 2-keto-3-deoxygluconate (KDG) to produce 2-keto-3-deoxy-6-phosphogluconate (KDPG). Therefore, suitable PGPR strains and inoculants thereof for the methods and composition disclosed herein may include a PGPR strain that expresses 2-dehydro-3-deoxygluconokinase. SEQ ID NO:13 provides the polynucleotide sequence encoding for 2-dehydro-3-deoxygluconokinase. SEQ ID NO:14 provides the amino acid sequence for 2-dehydro-3-deoxygluconokinase.

The exuR gene encodes a hexuronate utilization operon transcriptional repressor. Therefore, suitable PGPR strains and inoculants thereof for the methods and composition disclosed herein may include a PGPR strain that expresses a hexuronate utilization operon transcriptional repressor. SEQ ID NO:15 provides the polynucleotide sequence encoding for a hexuronate utilization operon transcriptional repressor. SEQ ID NO: 16 provides the amino acid sequence for a hexuronate utilization operon transcriptional repressor.

The exuT gene encodes a hexuronate transporter which exhibits hexuronate transmembrane transporter activity. Therefore, suitable PGPR strains and inoculants thereof for the methods and composition disclosed herein may include a PGPR strain that expresses a hexuronate transporter. SEQ ID NO: 17 provides the polynucleotide sequence encoding for a hexuronate transporter. SEQ ID NO: 18 provides the amino acid sequence for a hexuronate transporter.

In some embodiments, the PGPR strain may express one or more pectinase enzymes selected from a group consisting of pectin lyase (EC 4.2.2.10), pectate lyase (EC 4.2.2.2), polygalacturonase (EC 3.2.1.15), and pectin esterase (EC 3.1.1.11). SEQ ID NO:19 provides the polynucleotide sequence encoding for a pectate lyase precursor. SEQ ID NO:20 provides the amino acid sequence for a pectate lyase precursor. SEQ ID NO:21 provides the polynucleotide sequence encoding for a pectin-lyase like protein. SEQ ID NO:22 provides the amino acid sequence for a pectin-lyase like protein. SEQ ID NO:23 provides the polynucleotide sequence encoding for a pectin lyase. SEQ ID NO:24 provides the amino acid sequence for a pectin lyase.

“Percentage sequence identity” may be determined by aligning two sequences of equivalent length using the Basic Local Alignment Search Tool (BLAST) available at the National Center for Biotechnology Information (NCBI) website (i.e., “bl2seq” as described in Tatiana A. Tatusova, Thomas L. Madden (1999), “Blast 2 sequences—a new tool for comparing protein and nucleotide sequences”, FEMS Microbiol Lett. 174:247-250, incorporated herein by reference in its entirety). For example, percentage sequence identity between SEQ ID NO:1 and another sequence for comparison may be determined by aligning these two sequences using the online BLAST software provided at the NCBI website.

“Percentage sequence identity” between two deoxyribonucleotide sequences may also be determined using the Kimura 2-parameter distance model which corrects for multiple hits, taking into account transitional and transversional substitution rates, while assuming that the four nucleotide frequencies are the same and that rates of substitution do not vary among sites (Nei and Kumar, 2000) as implemented in the MEGA 4 (Tamura K, Dudley J, Nei M & Kumar S (2007) MEGA4: Molecular Evolutionary Genetics Analysis (MEGA) software version 4.0. Molecular Biology and Evolution 24:1596-1599), preferably version 4.0.2 or later. The gap opening and extension penalties are set to 15 and 6.66 respectively. Terminal gaps are not penalized. The delay divergent sequences switch is set to 30. The transition weight score is 35 set to 0.5, as a balance between a complete mismatch and a matched pair score. The DNA weight matrix used is the IUB scoring matrix where x's and n's are matches to any IUB ambiguity symbol, and all matches score 1.9, and all mismatched score O.

### Pectin and Pectin-Related Saccharides

The disclosed compositions and methods include or utilize pectin or pectin-derived sugars in order to sugars to enhance the efficacy of PGPR in regard to promoting plant growth and plant health. “Pectin” is a heteropolysaccharide found natively in the primary cell walls of terrestrial plants having a typical molecular weight of 60,000-130,000 g/mol, which varies based on the origin of the pectin and the extraction conditions. As used herein, “pectin” is meant to include extracted pectin that has been extracted from its native condition (e.g., extracted pectin from primary cell walls of terrestrial plants).

The compositions and methods disclosed herein may comprise and/or utilize a relatively high molecular weight polysaccharide such as a relatively high molecular weight pectin. In some embodiments, the compositions and methods disclosed herein comprise and/or utilize pectin having an average molecular weight of at least about 1000, 2000, 5000, 10000, 15000, 20000, 25000, 30000, 35000, 40000, 45000, 50000, 55000, or 60000 g/mol. Is some embodiments, the relatively high molecular weight polysaccharide represents at least about 10%, 20%, 30%, 40%, 50%, 60%, 70%, 80%, 90%, 95%, or higher of the total amount of carbohydrate in a composition as contemplated herein.

The disclosed composition and methods may include and/or utilize pecting and/or pectin derived sugars in any form. In some embodiments, the pectin and/or pectin derivated sugars are in powder form. The powder form may be utilized to prepare a solution of the pectin and/or pectin derivated sugars. Solutions of pectin prepared for use in the presently disclosed methods may have a concentration (w/w) of about 0.0001%, 0.0005%, 0.001%, 0.005%, 0.01%, 0.05%, 0.1%, 0.5%, 1%, 2%, 3%, 4%, or 5%, or solutions of pectin prepared for use in the presently disclosed methods may have a concentration of pectin within a range bounded by any of these percentage values (e.g., within a range of 0.01%-1%). A solution thus prepared may be utilized in the methods disclosed herein, including methods for treating plants, increasing nodulation in legumes, and/or treating animals.

The pectin or pectin-related saccharides utilized in the disclosed composition and methods may be isolated or substantially purified. The terms “isolated” or “substantially purified” refers to pectin or pectin-related saccharides that have been removed from a natural environment and have been isolated or separated, and are at least 60% free, preferably at least 75% free, and more preferably at least 90% free, even more preferably at least 95% free, and most preferably at least 100% free from other components with which they were naturally associated, which other components may include but are not limited to cellulose or other non-pectin polysaccharides.

Although the composition of pectin may vary among plants, pectin typically has a composition in which D-galacturonic acid is the main monomeric constituent (i.e., typically D-galacturonic acid represents >50% of the monomeric constituents of pectin). The D-galacturonic residues of pectin optionally may be substituted with D-xylose or D-apiose to form xylogalacturonan and apiogalacturonan, respectively, branching from a D-galacturonic acid residue. So-called “rhamnogalcturonan pectins” contain a backbone of repeating disaccharides of D-galacturonic acid and L-rhamnose. Pectins and pectin-derived products suitable for use in the presently disclosed compositions and methods may include pectin in which D-galacturonic acid represents >50% of the monomeric constituents of the pectin, optionally where one or more of the D-galacturonic residues of pectin are substituted with D-xylose or D-apiose to form xylogalacturonan and apiogalacturonan, respectively, branching from a D-galacturonic acid residue. Pectins and pectin-derived products suitable for use in the presently disclosed compositions and methods may include so-called “rhamnogalcturonan pectins” that contain a backbone of repeating disaccharides of D-galacturonic acid and L-rhamnose.

In nature, the majority of carboxyl groups of galacturonic acid in pectin are esterified with methanol (i.e., >50% and as much as 80% of the carboxyl groups of galacturonic acid in pectin are esterified with methanol). During extraction, this percentage may decrease where extraction may result in hydrolysis of the ester bond, and extracted pectins may be categorized as high-ester versus low-ester pectins having <50% of galacturonic acid residues being esterified. Non-esterified galacturonic acid units can be either free acids (i.e., carboxyl groups) or salts with sodium, potassium, or calcium (i.e., galacturonate salts). Pectins and pectin-derived products suitable for use in the presently disclosed compositions and methods may include pectins in which the majority of carboxyl groups of galacturonic acid in pectin are esterified with methanol (i.e., >50% and as much as 80% of the carboxyl groups of galacturonic acid in pectin are esterified with methanol). After extraction, pectins and pectin-derived products suitable for use in the presently disclosed compositions and methods may include extracted pectins (e.g., high-ester pectins or low-ester pectins having <50% of galacturonic acid residues being esterified).

In nature, D-galacturonic acid may be synthesized from D-gluconoric acid derivatives (e.g., from UDP-D-glucuronate via 4-epimerization) and conversely, D-galacturonic acid in pectin may be metabolized to form D-gluconoric acid derivatives (e.g., 5-dehydro-4-deoxy-D-glucuronate via oligogalacturonate lysis). As used herein, pectin-related saccharides include pectin-derived saccharides such as hydrolyzed pectin, D-galacturonic acid (or D-galacturonate salts), and D-gluconoric acid (or D-gluconorate salts), polymers thereof, or combinations thereof.

The compositions and methods disclosed herein may include or utilize a saccharide that is a substrate for an enzyme or transporter encoded by a gene selected from the group consisting of uxaA (altronate dehydratase), uxaB (altronate oxidoreductase), uxaC (uronate isomerase), uxuA (mannonate dehydratase), uxuB (D-mannonate oxidoreductase), kdgA (4-hydroxy-2-oxoglutarate aldolase), kdgK (2-dehydro-3-deoxygluconokinase), exuR (hexuronate utilization operon transcriptional repressor), exuT (hexuronate transporter), and combinations thereof. The compositions and methods disclosed herein may include or utilize a saccharide that is a substrate for a pectinase enzyme (e.g., a pectinase enzyme selected from a group consisting of pectin lyase, pectate lyase, polygalacturonase, and pectin esterase).

Substrates as such may include but are not limited to saccharides derived from pectin such as D-galacturonate and D-glucuronate. The saccharide may comprise a mixture of sugars or the saccharide may comprise a heteropolysaccharide. In embodiments in which the saccharide is a heterogeneous mixture of sugars or the saccharide is a heteropolysaccharide, preferably D-galacturonate monomeric units, D-glucuronate monomeric units, or the sum of D-galacturonate monomeric units and D-glucuronate monomeric units represent >50%, >60%, >70%, >80%, >90%, or >95% of total monomeric units in the heterogeneous mixture of sugars or the heteropolysaccharide.

The disclosed pectin and pectin-related substances may include synthetic pectin. Synthetic pectin may include pectin synthesized by polymerizing pectin monomers (e.g., uronic acid) in vitro to form pectin-like substance referred to as synthetic pectin. (See, e.g., U.S. Pat. No. 2,156,223. Furthermore, the disclosed pectin and pectin-related substances may include naturally and non-naturally occurring polyuronic acids.

In the disclosed methods and compositions, pectin may be present at a desirable concentration, for example, in soil surrounding a plant, in a seed coating, or in animal feed. When pectin is administered to soil, the pectin may be administered, for example, to achieve a concentration in soil (w/w) of about 0.0001%, 0.0005%, 0.001%, 0.005%, 0.01%, 0.05%, 0.1%, 0.5%, 1%, 2%, 3%, 4%, or 5%, or to achieve a concentration in soil within a concentration range bounded by any of these percentage values (e.g., within a range of 0.001%-0.01%). When pectin is present in a seed coating, the pectin may be present at a concentration (w/w) in the coating of about 0.001%, 0.005%, 0.01%, 0.05%, 0.1%, 0.5%, 1%, 5%, 10%, 20%, 30%, 40%, 50%, or higher, or the pectin may be present at concentration (w/w) in the coating within a concentration range bounded by any two of these values (e.g., within a range of 0.1%-1%). When pectin is present in animal feed, the pectin may be present at a concentration (w/w) in the animal feed of about 0.005%, 0.01%, 0.05%, 0.1%, 0.5%, 1%, 2%, 3%, 4%, 5%, 10% or higher or the pectin may be present in the animal feed at within range concentration bounded by any two of these values (e.g., within a range of 0.1%-1%).

### Inoculants

The presently disclosed PGPR may be formulated as an inoculant for a plant. The term “inoculant” means a preparation that includes an isolated culture of a PGPR and optionally a carrier. Inoculants comprising PGPR and carriers are known in the art. (See, e.g., Bashan, “Inoculants of Plant Growth-Promoting Bacteria for use in Agriculture,” Biotechnology Advances, Vol. 16, No. 4, pp. 729-770, 1998). PGPR inoculants may be administered to plants (e.g. to the roots of plants), to seeds (e.g., as a coating for the seed or at the time that the seed is planted), or to soil (e.g., to soil surrounding plants to be treated).

A PGPR inoculant may be described as a formulation containing one or more PRPR species in a carrier material, which may be an organic carrier, an inorganic carrier, or a carrier synthesized from defined molecules. Optionally, the carrier may be sterile or sterilized prior to be formulated with the PGPR to form the PGPR inoculant. Preferably, the carrier is nontoxic, biodegradable and nonpolluting. In the disclosed inoculants comprising a pectin saccharide, the pectin saccharide optionally may function as a carrier or optionally the inoculants may comprise a carrier other than the pectin saccharide.

The carrier of the PGPR inoculant is the delivery vehicle for the live PGPR to the plant, seeds, or soil. The carrier represent is the major portion by volume or weight of the inoculant. Suitable carriers may include liquids, powders (e.g., having an average effective particle diameter of 0.075 to 0.25 mm), granulars (e.g., having an average effective particle diameter of 0.35 to 1.18 mm), and slurries which have the capacity to deliver a sufficient number of viable PGPR cells to the plant, seeds, or soil. Preferably, the carrier extends the shelf-life of the PGPR (e.g., such that the PGPR has a shelf-life of at least 1 or 2 years at room temperature). Examples of carriers include but are not limited to peat, coal, clays, inorganic soil material, plant waste materials, composts, farmyard manure, soybean meal, soybean oil, peanut oil, wheat bran, inert materials such as vermiculite, perlite, phosphate, polyacrylamide, alginate beads, oil-dried bacteria. In some embodiments, the PGPR may be encapsulated by a carrier, for example, where the carrier is a carbohydrate that forms a matrix around the PGPR.

The PGPR utilized in the disclosed composition and methods may be isolated or substantially purified. The terms “isolated” or “substantially purified” refers to PGPR that have been removed from a natural environment and have been isolated or separated, and are at least 60% free, preferably at least 75% free, and more preferably at least 90% free, even more preferably at least 95% free, and most preferably at least 100% free from other components with which they were naturally associated. An “isolated culture” refers to a culture of the PGPR that does not include significant amounts of other materials such as other materials which normally are found in soil in which the PGPR grows and/or from which the PGPR normally may be obtained. An “isolated culture” may be a culture that does not include any other biological, microorganism, and/or bacterial species in quantities sufficient to interfere with the replication of the “isolated culture.” Isolated cultures of PGPR may be combined to prepare a mixed culture of PGPR.

The inoculant typically includes a suitable amount of PGPR relative to carrier. In some embodiments, the inoculant includes 10²-10¹² cfu PGPR per ml carrier (or per gram carrier), or 10⁴-10¹⁰ cfu PGPR per ml carrier (or per gram carrier), or 10⁶-10⁸ cfu PGPR per ml carrier (or per gram carrier). The composition may include additional additives including buffering agents, surfactants, adjuvants, or coating agents. Suitable carriers may include, but are not limited to, water or other aqueous solutions, slurries, solids (e.g., peat, wheat, bran, vermiculite, and pasteurized soil) or dry powders.

In the disclosed methods and compositions, PGPR may be present at a desirable concentration, for example, in soil surrounding a plant, in a seed coating, or in animal feed. In some embodiments where PGPR is applied to soil, PGPR may be applied as a seedling root-dip or as a soil drench at a concentration of about 10²-10¹² cfu/ml, 10⁴-10¹⁰ cfu/ml, or about 10⁶-10⁸ cfu/ml. In some embodiments where PGPR is present as a coating on a seed, suitable application concentrations may be between 10²-10⁸ cfu per seed, preferably 10⁴-10⁷ cfu per seed. In some embodiments where PGPR is present in animal feed, the PGPR may be presented at a concentration of at least about 10⁴ CFU/g of feed. More preferably, the spore-forming strain of the genus Bacillus is present in the composition at a concentration of at least about 10⁵ CFU/g of feed. Even more preferably, the spore-forming strain of the genus Bacillus is present in the composition at a concentration of at least about 10⁶ CFU/g of feed or per ml of water or at least about 10⁷ CFU/g of feed or per ml of water. A suitable concentration range may include 10⁴-10⁷ CFU/g of feed or per ml of water or sub-ranges there within.

The disclosed inoculants and compositions may include additional agents for promoting plant growth and health, including additional agents for promoting nodulation. The additional agents may include additional bacterial inoculants, including, but not limited to, additional rhizobacteria such as a nitrogen-fixing bacteria and/or a phosphate-solubilizing bacteria. Additional agents may include fungal inoculants, for example mycorrhizae. Additional agents may include plant nutrients such as nitrogen salts and/or phosphate salts and/or potassium salts.

### Methods of Treating Plants, Seeds, or Soil

Also disclosed are methods of using pectin or pectin-related saccharides to improve the efficacy of PGPR in regard to promoting growth or health in plant. The disclosed methods for promoting growth or health in plant may include, but are not limited to, methods of increasing nodulation in legumes. The disclosed methods may include administering the above-described inoculants comprising a PGPR and a pectin saccharide to plants, seeds, or soil. In some embodiments, the disclosed methods for improving plant growth or plant health may include: (a) treating plants, seeds, or soil with a plant growth promoting rhizobacteria (PGPR) that expresses a protein associated with pectin metabolism and (b) treating the plants, seeds, or soil with a saccharide comprising pectin or a pectin-related saccharide (e.g., hydrolyzed pectin, D-galacturonate, D-glucuronate, or mixtures thereof), where the plants, seeds, or soil may be treated with the PGPR and the saccharide concurrently or in either order (i.e., the PGPR may be administered before, concurrently with, or after the saccharide is administered). The PGPR and pectin saccharide may be formulated as an inoculant and administered concurrently to treat plants (e.g., administered to the roots of plants), to seeds (e.g., as a coating for seeds), or to soil (e.g., as a soil amendment).

The disclosed methods may be utilized to improve plant growth or plant health by controlling soil-borne pests. Soil-borne pests controlled by the disclosed methods may include but are not limited to nematodes and herbivorous insects. The disclosed methods may be utilized to improve plant growth or plant health by controlling or treating a disease. Disease controlled or treated by the disclosed methods may include but are not limited to a bacterial disease, a fungal disease, and a viral disease.

The presently disclosed PGPR and pectin saccharide may be administered as an inoculant for treating plants. The methods of treatment contemplated herein may include treating a plant directly including treating leaves, stems, or roots of the plant directly. The methods of treatment contemplated herein may include treating seeds of the plant, e.g., coating the seeds prior to the seeds being planted to produce a treated plant. The methods contemplated herein also may include treating a plant indirectly, for example, by treating soil or the environment surrounding the plant (e.g., in-furrow granular or liquid applications). Suitable methods of treatment may include applying an inoculant including the PGPR and the saccharide via high or low pressure spraying, drenching, and/or injection. Plant seeds may be treated by applying low or high pressure spraying, coating, immersion, and/or injection. After plant seeds have been thusly treated, the seeds may be planted and cultivated to produce plants. Plants propagated from such seeds may be further treated with one or more applications. Suitable application concentrations may be determined empirically. In some embodiments where the PGPR and pectin saccharide are applied as a spray to plants, suitable application concentrations may include spraying 10⁶-10¹⁸ colony forming units (cfu) per hectare of plants, more commonly 10⁷-10¹⁵ cfu per hectare. For coated seeds, in some embodiments, suitable application concentrations may be between 10²-10⁸ cfu per seed, preferably 10⁴-10⁷ cfu per seed. In other embodiments, the PGPR and pectin saccharide may be applied as a seedling root-dip or as a soil drench at a concentration of about 10²-10¹² cfu/ml, 10⁴-10¹⁰ cfu/ml, or about 10⁶-10⁸ cfu/ml.

### Methods of Treating Animals

Bacillus species isolates cultured from plant rhizospheres have the ability to utilize complex plant polysaccharides as a carbon and energy source. Increasingly, animal feeds are plant based and many of the plant-derived polysaccharides and other compounds (e.g. phytic acid) are not readily degraded or utilized by fish, poultry or livestock. In fact, in many cases these plant-derived compounds such as phytic acid serve as an anti-nutrient that can make animals anemic. Using Bacillus or other species that can degrade complex plant polysaccharides can promote feed conversion efficiency and these rhizosphere isolates are ideally suited to help improve animal feeds and degrade phytic acid to improve animal nutrition.

As such, also disclosed are methods of using pectin or pectin-related saccharides to improve the efficacy of PGPR in regard to promoting growth or health in animals. The disclosed methods may include administering the afore-described inoculants comprising a PGPR and a pectin saccharide to animals (e.g., in the form of an animal feed composition such as a pelleted feed composition comprising the afore-described inoculants). In some embodiments, the disclosed methods for improving animal growth or animal health may include: (a) administering to an animal a plant growth promoting rhizobacteria (PGPR) that expresses a protein associated with pectin metabolism and (b) administering to the animal a pectin saccharide comprising pectin or a pectin-related saccharides (e.g., hydrolyzed pectin, D-galacturonate, D-glucuronate, or mixtures thereof), where the animals may be administered the PGPR and the pectin saccharide concurrently or in either order (i.e., the PGPR may be administered before, concurrently with, or after the saccharide is administered).

Feed compositions comprising the PGPR and pectin saccharide may be administered to animals orally. Oral administration includes, but is not limited to, delivery in feed, water, by oral gavage or aerosol spray. If supplied in an animal feed, the feed may comprise between 10⁴ and 10⁹ cfu PGPR/gm of finished feed. Suitably the feed comprises between 10⁵ and 5×10⁷ cfu PGPR/gm feed. The PGPR and pectin saccharide may be added to the feed during production, after production by the supplier, or by the person feeding the animals, just prior to providing the food to the animals.

An animal feed composition may be prepared by forming a mixture of the animal feed and an inoculant as discussed above, and then optionally forming a compressed or pelleted animal feed from the mixture. Animal feed suitable for preparing animal feed compositions as disclosed herein may include animal feed comprising plant material (e.g., hay straw, silage, grains (e.g., maize, soybean, wheat, oats, barley, sorghum, and rice), and legumes). Animal feed suitable for preparing animal feed compositions as disclosed herein amy include fish products (e.g., fish oils and fish proteins).

The disclosed methods for promoting growth or health in animals may be practiced in order to increase overall gastrointestinal health, improve production performance, and reduce enteric bacterial pathogens of importance to both animal health and human food safety. These PGPR and pectin saccharide may be added to animal diets at the rate of about 10⁴ to 10⁹ PGPR per gram of finished feed for optimal inclusion rate, if the bacteria or probiotic compositions being administered continuously, and a higher inclusion rate may be necessary if the PGPR or the compositions are provided intermittently. While administration though the feed is a preferred route of administration, the PGPR and pectin saccharide may also be administered via drinking water, through course spray, through aerosol spray, or through any other means by which the agricultural animals may ingest these PGPR and pectin saccharide.

The disclosed methods may include methods of promoting growth or health in aquatic animals, which may include farmed fish (e.g. catfish or tilapia) and crustaceans (e.g., shrimp). The term “catfish” refers to a fish belonging to the genus Ictaluri. Catfish may include the species Ictaluri punctatus Rafinesque.

### Methods for Preparing the Disclosed Compositions and Inoculants

Also disclosed are methods of using pectin or pectin-related saccharides to prepare compositions and inoculants as disclosed herein. The methods may include combining PGPR and pectin, which has been extracted from pectin-containing plant material, or pectin-related saccharides to prepare the disclosed compositions and inoculants. Optionally, a carrier may be combined with the PGPR and pectin or pectin-related saccharides to prepare the disclosed compositions and inoculants.

In some embodiments, the methods may include combining 10²-10¹² cfu PGPR per ml carrier (or per gram carrier), or 10⁴-10¹⁰ cfu PGPR per ml carrier (or per gram carrier), or 10⁶-10⁸ cfu PGPR per ml carrier (or per gram carrier). In some embodiments, the methods may include combining pectin, which has been extracted from pectin-containing plant material, or pectin-related saccharides may be combined with PGPR and optionally a carrier to prepare the disclosed compositions and inoculants, wherein the pectin or pectin-related saccharides are present in the prepared compositions and inoculants at a concentration of at least about 0.1%, 0.5%, 1.0%, 1.5%, or 2.0% (w/w or w/v) to about 0.5%, 1.0%, 1.5%, 2.0%, or 5.0% (w/w or w/v). In some embodiments, the methods may include combining PGPR and pectin at a concentration of about at least about 10², 10³, 10⁴, 10⁵, 10⁶, 10⁷, 10⁸, 10⁹, 10¹⁰, 10¹¹, 10¹², 10¹³, or 10¹⁴ cfu PGPR per gram pectin or pectin-related saccharides, to about 10³, 10⁴, 10⁵, 10⁶, 10⁷, 10⁸, 10⁹, 10¹⁰, 10¹¹, 10¹², 10¹³, 10¹⁴, 10¹⁵ cfu PGPR per gram pectin or pectin-related saccharides (e.g., ranges such as 10⁷ to 10¹² cfu PGPR per gram pectin or pectin-related saccharides are contemplated herein). In the methods, additional additives including buffering agents, surfactants, adjuvants, and coating agents may be combined with the PGPR, pectin or pectin-related saccharides, and optional carrier in order to prepare the disclosed compositions and inoculants. Compositions and inoculants prepared by the afore-disclosed methods also are contemplated herein.

### EXAMPLES

The following Examples are illustrative and are not intended to limit the scope of the claimed subject matter.

### Example 1-Deciphering the Conserved Genetic Loci Implicated in Plant Disease Through Comparative Genomics of Bacillus amyloliquefaciens Subsp. Plantarum (Now Bacillus velezensis) Strains

Reference is made to Hossain et al., “Deciphering the conserved genetic loci implicated in plant disease through comparative genomics of Bacillus amyloliquefaciens subsp, plantarum [(now Bacillus velezensis)] strains,” Frontiers in Plant Science, 2015 Aug. 17; 6:631 doi: 10.3389/fpls.2015.00631. eCollection 2015, (hereinafter referred to as “Hossain et al., Frontiers Plant Science 2015), the content of which is incorporated herein by reference in its entirety.

We claim:

1. A method for controlling pests of a plant, the method comprising (a) treating the plant, seeds of the plant, or soil surrounding the plant with Bacillus velezensis (BV) and (b) treating the plant, the seeds of the plant, or the soil surrounding the plant with a saccharide comprising pectin or a pectin-related saccharide, wherein the pectin-related saccharide is a heteropolysaccharide comprising D-galacturonate monomers which represent at least 50% of all monomers of the heteropolysaccharide.

2. The method of claim 1, wherein the saccharide is pectin having an average molecular weight of at least about 30000 g/mol.

3. The method of claim 1 comprising treating the plant with the BV and treating the plant with the saccharide.

4. The method of claim 1 comprising treating the seeds of the plant with the BV and treating the seeds of the plant with the saccharide.

5. The method of claim 1 comprising treating the soil surrounding the plant with the BV and treating the soil surrounding the plant with the saccharide.

6. The method of claim 5, further comprising treating soil surrounding the plant with a nitrogen-fixing bacteria.

7. The method of claim 5, wherein treating the soil with BV comprises administering the (BV) to the soil surrounding the plant at a rate that delivers 10\hat{ }3-10\hat{ }5 CFU per gram soil.

8. The method of claim 1, wherein the plant, the seeds of the plant, or the soil surrounding the plant are treated concurrently with the BV and the saccharide.

9. The method of claim 1, wherein the plant, the seeds of the plant, or the soil surrounding the plant are treated first with the (BV) and subsequently the plant, the seeds of the plant, or the soil surrounding the plant are treated with the saccharide.

10. The method of claim 1, wherein the plant, the seeds of the plant, or the soil surrounding the plant are treated first with the saccharide and subsequently the plant, the seeds of the plant, or the soil surrounding the plant are treated with the (BV).

11. The method of claim 1, wherein the pests are soil-borne pests.

12. The method of claim 1, wherein the pests are nematodes.

13. The method of claim 1, wherein the pests are herbivorous insects.

14. The method of claim 1, wherein the plants are cotton plants.

15. The method of claim 1, wherein the plants are soybean plants.


SEQUENCE LISTING (26 sequences)

SEQ ID NO: 1 - DNA, 759 bp, Bacillus amyloliquefaciens
  source 1..759: mol_type = genomic DNA
atgaaagaggtcataaaaatccataaaaacgataacgtgcttctcgccatgcgccattttaacaaaggggagcggctgcatacagacggactgacgattgaagtgaaggaccctgtaaagcgggggcataaaatcgcgctgcaaaccattgaagaaaacggcagcatcattaaatacggtttttcaatcgggcgtgcgacaaggcggatttcggccggcgagcatattcatacccataatctgcaaacgaatttatccgatgtgcaagaatacacgtattcaccgcgcttcgaggacaatccttttacaaatgagaaccggacatttaaaggctacagaagagaaaacggtacatccggcgttcggaatgaactgtggatcgtgcctacagtcggctgcgtaaacggcgtcgctgagaaaatcctgcagcgtttcgtaaaagaggcgaaagacattgcgccctttgacaatgtgctggttttaaagcaccaatacggctgctcccagcttggcgatgatcatgaaaatacgaaacaaatgctgatgaacgccatccgtcatccaaatgcgggcggtgtgctggttttagggctcggctgtgaaaataatgagctggcggcgattagggaaacgctttctgaagtgaacggagatcgggtgaaatttctcgaatcacaagctgtcacagctgagactgacagcagatcaactcgaaatgcgtctaagacttcttggcatttcaaatataaacacaattaa

SEQ ID NO: 2 - protein, 252 aa, Bacillus amyloliquefaciens
MKEVIKIHKNDNVLLAMRHFNKGERLHTDGLTIEVKDPVKRGHKIALQTIEENGSIIKYGFSIGRATRRISAGEHIHTHNLQTNLSDVQEYTYSPRFEDNPFTNENRTFKGYRRENGTSGVRNELWIVPTVGCVNGVAEKILQRFVKEAKDIAPFDNVLVLKHQYGCSQLGDDHENTKQMLMNAIRHPNAGGVLVLGLGCENNELAAIRETLSEVNGDRVKFLESQAVTAETDSRSTRNASKTSWHFKYKHN

SEQ ID NO: 3 - DNA, 1446 bp, Bacillus amyloliquefaciens
  source 1..1446: mol_type = genomic DNA
ttgcggaagctgaataaaaagatgtgcggtgaatacaatcaatatcctgaaaaagtccttcagttcggcgaaggcaactttttgcgggccttcgcagactggcagatcgatcaattgaatcaactgacggattttaacggaagcgttgtcgccgtacagccgagaggctctgagaaaatcaaacgtttaaacgagcaggacggtttatttacgctttttttgcaggggataaaggacggaaaaccggctgaagaacacatgatcgtcaaatccatcagccgcggcattgatcttttttctgactatgattcctttaaacggctggcagcacaagaggagcttcgttttatcatttccaatacgacagaagcgggaatcacgtttgagaaaggcgaccggcttgaggatagacctcaaaaaacattccccgggaaattagcggcgttcctttatttccgctataatgcatttgcgggtgacgccgagaaagggtgcattgtgcttccatgtgaactggtcgaagaaaacggacggaaactgaaggcggccgtgcttcaatatgccgggctgtgggagctggaacctggttttactcaatggattcatgacgcgaacatcttctgcaataccctcgtggatcggattgttcccggatttcctgttgataccgcggaagaacttaccgattttctcggatacgaagaccgtctgctggtggttggtgagcattattatttgtgggtgatagaagggccggaacagcttcagaatgagcttccttttgctgaggccgggctgaacgcactgatcacgtctgaccttacgtcttacaggacaaaaaaggtgagaatcttaaacggcgctcacacggcattggcgccagtggctttactatacggactgcaaaccgtccgggaagcggctgagcacgaggtgacgggcagattcattgaagagctgatcaatgaagaaatcctccctgtgctgcaaatggagggtgtcacccaatacgcggcagatgtgttacagcggtttaaaaacccttatatccaccattatctgcaaagcattacccttaacgccgccgcgaaattcaaaacaagaaacctgccgacgctgaaggcttatatcgaacaggagggccggctacctgagaagctggtattttcgttcagcgccatgatctattcatactggaacggaccgaacaaaccggctgacggcgagggggttcttaaccgttttcaaatggcgcggtcccgttgcaaagatgatatgtttcaagcggcatctgccatattaggagaagaccggttgtggggaaagaatctgaaccatcttccggggctgactgaacgaacggctttttttctgtctgtcattcataagcggggtatgaagcatgcgttacatgaatgctgcggaaaaaagggggaagtgaaatga

SEQ ID NO: 4 - protein, 481 aa, Bacillus amyloliquefaciens
MRKLNKKMCGEYNQYPEKVLQFGEGNFLRAFADWQIDQLNQLTDFNGSVVAVQPRGSEKIKRLNEQDGLFTLFLQGIKDGKPAEEHMIVKSISRGIDLFSDYDSFKRLAAQEELRFIISNTTEAGITFEKGDRLEDRPQKTFPGKLAAFLYFRYNAFAGDAEKGCIVLPCELVEENGRKLKAAVLQYAGLWELEPGFTQWIHDANIFCNTLVDRIVPGFPVDTAEELTDFLGYEDRLLVVGEHYYLWVIEGPEQLQNELPFAEAGLNALITSDLTSYRTKKVRILNGAHTALAPVALLYGLQTVREAAEHEVTGRFIEELINEEILPVLQMEGVTQYAADVLQRFKNPYIHHYLQSITLNAAAKFKTRNLPTLKAYIEQEGRLPEKLVFSFSAMIYSYWNGPNKPADGEGVLNRFQMARSRCKDDMFQAASAILGEDRLWGKNLNHLPGLTERTAFFLSVIHKRGMKHALHECCGKKGEVK

SEQ ID NO: 5 - DNA, 1206 bp, Bacillus amyloliquefaciens
  source 1..1206: mol_type = genomic DNA
atgagggccaacggcattgaagagcgctgcattaccggaaacgcttcagatgaagagaagtttttcgcctgggcaaaaacggtgccgatggcgataggcaatccgctgtacagctggacgcatttagagctgcagcgctggttcggcatttatgacgtattgaatgaaaaaacggccgcggcaatttggaaaaaaacgaatgagctcctgcagggagacgggtttggcgcaagggatctcattctgaaatcaaaagtcaaagtcatttgcacaaccgacgatcctgccgatgcgctgacataccacgaactgttgaaagaaagtgattttccggttcaagtgctgcccggatttcggcctgacaaaggactggacatcagcagcccgggttttgccgattgggtccgttcgctggagagcgcttccggaatagctgtcaccacctaccaatcgtatttagatgcgttagaatcccgcgtccgcttttttcacaatgccgggggaagagtgtcggatcatgcgttagatcaaatggtttatgccgagacgaccgaagaggaagcggcccggatttttgccgccggattaagcggagagcatgtgtcttttgaagatgaaaagaaattcaaaacacggacgctgcaatatctttgcggtctttacgccgaactggactgggcgatgcagttccatattaacgcgttaagaaataccaatacaaataagttcagcagcctcggtcctgatacgggatatgactcaattaatgatgaacgtatcgcaaagccgcttgcccggctgttagattccgccgaaaaaaagcggcaattgcctaaaacgattttgtattctttgaatccgaatgacaactatattatcgccagcatgatcaacagctttcaggatgggaaaacaccggggaaaattcaattcggcacagcctggtggttcaatgacacgaaagatggaatgcttcagcaaatgaaagcgttatcaaacatgggcctgttcagccgttttatcgggatgctgacggattcaagaagtttcctgtcctacccccgccatgaatacttcaggcggcttgtctgcacgctgatcggcggctgggcggaacaaggcgaagcgccttacgatatggagcttttaggaaggattgtcgagggaatttgttaccggaatgctgaggaatatttccgcttttaa

SEQ ID NO: 6 - protein, 401 aa, Bacillus amyloliquefaciens
MRANGIEERCITGNASDEEKFFAWAKTVPMAIGNPLYSWTHLELQRWFGIYDVLNEKTAAAIWKKTNELLQGDGFGARDLILKSKVKVICTTDDPADALTYHELLKESDFPVQVLPGFRPDKGLDISSPGFADWVRSLESASGIAVTTYQSYLDALESRVRFFHNAGGRVSDHALDQMVYAETTEEEAARIFAAGLSGEHVSFEDEKKFKTRTLQYLCGLYAELDWAMQFHINALRNTNTNKFSSLGPDTGYDSINDERIAKPLARLLDSAEKKRQLPKTILYSLNPNDNYIIASMINSFQDGKTPGKIQFGTAWWFNDTKDGMLQQMKALSNMGLFSRFIGMLTDSRSFLSYPRHEYFRRLVCTLIGGWAEQGEAPYDMELLGRIVEGICYRNAEEYFRF

SEQ ID NO: 7 - DNA, 1077 bp, Bacillus amyloliquefaciens
  source 1..1077: mol_type = genomic DNA
atgaatatcacgtttcgctggtacggccaaggtaatgatacggtgactcttgaacatgtcagacaaattcccggggtcaaaggcatcgtctgggctcttcaccagaagcacgcgggcgaagtatgggaaaaggaggacattgaagcggaagtcagatacattcaatcctatgggtttcatgcagaggtcgtggaaagcgtgaatgttcatgaagccattaaactcggtcatgaagaacgcggacaatatattgagcactataaacagaccattcgcaatctgtccgaattcggcgtgaaagtcatctgctacaatttcatgccgatatttgactggacgcgtaccgatatgttccgcgcgctggaggatgggtcaaccgcactgttttttgaaaaagcgaaagtggaaagtctccctccggaggaactgatccgcacggttgaagaatcttctgatttgacgcttcccgggtgggagccggagaaaatggccaggatcaaggaactgtttgaggcctatcaaacggttgatgaaaaacagctttgggccaatcttgagttctttttacatgaaatccttcctgtagcggaggaacacggcattcaaatggccattcatccggatgatccgccctggtcaatttttggcctgccccgtattatcaccggtgagaaaagttatgaaaagctgcaagagatttcagattcacccgccaactgtattacgttgtgcacgggttcgatgggagcggatcccgccaatgatatggtgaagatcaccaaacactatgcgggagctgcgccgtttgcccatatacgaaatgtgaaaatctgcgataacggtgattttacggaaacttcgcatcttaccagtgatgggtccattgatattaccggtgttgtaaaagaattgcacggccaagactataaaggatatgtacggcctgatcacggccgtcatatctggggcgaacaatgccggccgggttatggtttatatgatcgtgcattaggaattatgtatttgaacggactgtgggatgcgttttcgtctcatgagaaaggggtgaagagatga

SEQ ID NO: 8 - protein, 358 aa, Bacillus amyloliquefaciens
MNITFRWYGQGNDTVTLEHVRQIPGVKGIVWALHQKHAGEVWEKEDIEAEVRYIQSYGFHAEVVESVNVHEAIKLGHEERGQYIEHYKQTIRNLSEFGVKVICYNFMPIFDWTRTDMFRALEDGSTALFFEKAKVESLPPEELIRTVEESSDLTLPGWEPEKMARIKELFEAYQTVDEKQLWANLEFFLHEILPVAEEHGIQMAIHPDDPPWSIFGLPRIITGEKSYEKLQEISDSPANCITLCTGSMGADPANDMVKITKHYAGAAPFAHIRNVKICDNGDFTETSHLTSDGSIDITGVVKELHGQDYKGYVRPDHGRHIWGEQCRPGYGLYDRALGIMYLNGLWDAFSSHEKGVKR

SEQ ID NO: 9 - DNA, 477 bp, Bacillus amyloliquefaciens
  source 1..477: mol_type = genomic DNA
atggacgaaacggggttttccagcgtattttcaaccaactttaccggtgcatttctggcatcgcaagtgtttggaaaagaccttttgaaggcggaatgtcccgcgattattaatatttcttctatgagtgcgtatacaccgatgacaaaggtgcccgcctacagcgcggccaaggcggcggttcagaattttacgatgtggatggctgttcatttcgcaaaaaaagggctgcgggtgaatgccatcgctccgggtttctttttgacagcgcaaaaccgtgagttgttattacaggaagatggaagtctgacagaccgatccaataagattatggcacatacaccgatgaaacgtttcgggaaaccggaagaccttctgggaacgctcctgtggctttcggatgattcatgctcaggcttcgtcaccggggtcaccgttcccgtcgacggaggttttatggcttattccggagtgtga

SEQ ID NO: 10 - protein, 158 aa, Bacillus amyloliquefaciens
MDETGFSSVFSTNFTGAFLASQVFGKDLLKAECPAIINISSMSAYTPMTKVPAYSAAKAAVQNFTMWMAVHFAKKGLRVNAIAPGFFLTAQNRELLLQEDGSLTDRSNKIMAHTPMKRFGKPEDLLGTLLWLSDDSCSGFVTGVTVPVDGGFMAYSGV

SEQ ID NO: 11 - DNA, 636 bp, Bacillus amyloliquefaciens
  source 1..636: mol_type = genomic DNA
atgctgcctaagtacgccatttacgaaaaacttctgcatgcacaagtggtggctgtcataaggggacaaaacagccaagaggcgcttgaagtttcaaaagcggccatttcaggaggtatcagcgccattgagcttacatacacaactcctgaagtggaagatgtctttaaggaactccggcatcaagatattcttcttggagccggctctgtaatggatacagaaacggcgagacacgccattttatcaggcgcctcatttatcgtcagctctcattttgtaaaagagatagcatctctatgcaaccagtacagcgtaccgtatcttccgggatgcatgagtgtgtctgatatggctttggcgctcgaagccggatgtgatgtggtaaagctgttccccgctaattcgtttgacccttcatttatcaagtcggtcaacggccctctgccgaatgtccgcattatgccgaccgggggtatctcattaaacagcatgaatgactggctctctgccggagcggtcgcggtcggtgtgggaagcgacttgacaaaagcatatcaaaagggcggctatgaggctgccgtatccctcagcaaagaatacgtctgccgaaaaaacgaatatacgggggtgtaa

SEQ ID NO: 12 - protein, 211 aa, Bacillus amyloliquefaciens
MLPKYAIYEKLLHAQVVAVIRGQNSQEALEVSKAAISGGISAIELTYTTPEVEDVFKELRHQDILLGAGSVMDTETARHAILSGASFIVSSHFVKEIASLCNQYSVPYLPGCMSVSDMALALEAGCDVVKLFPANSFDPSFIKSVNGPLPNVRIMPTGGISLNSMNDWLSAGAVAVGVGSDLTKAYQKGGYEAAVSLSKEYVCRKNEYTGV

SEQ ID NO: 13 - DNA, 957 bp, Bacillus amyloliquefaciens
  source 1..957: mol_type = genomic DNA
gtggcttctgattatggcatttttacgatcggtgatgcgatgatcaccttaaatcccgcaagcacaggacctttgcgttttgttacccattttgaacgaaaagcaggaggagctgaactgaattttgcgattgcatgcgccagattgaatctgcactcaaagtgggtcagcagactggggaaggatgagttcggcagagtgatttatcagtttgccagaggagaaggaattgatgtgactgacgtctcctatgttgaaggctttcctacatctttaaatgtcaaagagatcagaaatgacggttcaggcaagacgttttattatcgtttcaattcaccaattctgactcttgcaccggaagatataacagagaatatgtttgacggcattcacatcgttcatcttaccggtgtgttcctcgcgcttgataagaaaaatcttgaaattgctgaaagggtgctttccatcgccaaagcaagggggattcccgtatcttttgatccgaatatcaggctgaagctgtggacgattgaggaggcgcgtgcggcatttcatcaaatctttccgcatactgatattcttctggcgggccgggacgaaatgaaacagctgacaggtataggggaaaatgaagcattagcccggtttgcagagacctattcaatcagtcagcttgtcataaaagacggtgaagcaggttctgcgctgtatcatgatcataagtggattcacaaagaggctttcccggttgcgcctgttgacacggtaggagcgggtgacggctttaatgccggctacctgtacagttatctgcatggttttgaaccggaaaaacgtctggagtttgccaatgcggtcggtgcgctcgtcaccacagtttccggtgacaatgaagggcttccctatctagaagaggttctgtcatttgtaaacaaagagactgtgattgaacggtag

SEQ ID NO: 14 - protein, 318 aa, Bacillus amyloliquefaciens
MASDYGIFTIGDAMITLNPASTGPLRFVTHFERKAGGAELNFAIACARLNLHSKWVSRLGKDEFGRVIYQFARGEGIDVTDVSYVEGFPTSLNVKEIRNDGSGKTFYYRFNSPILTLAPEDITENMFDGIHIVHLTGVFLALDKKNLEIAERVLSIAKARGIPVSFDPNIRLKLWTIEEARAAFHQIFPHTDILLAGRDEMKQLTGIGENEALARFAETYSISQLVIKDGEAGSALYHDHKWIHKEAFPVAPVDTVGAGDGFNAGYLYSYLHGFEPEKRLEFANAVGALVTTVSGDNEGLPYLEEVLSFVNKETVIER

SEQ ID NO: 15 - DNA, 1002 bp, Bacillus amyloliquefaciens
  source 1..1002: mol_type = genomic DNA
atgaaaaccattacaatttctgatgtggcgaaatatgcaaacgtatctaaaagcaccgtatctcaatttttaaatcagcgctatgactatatgagcgagaaaacgaaacaaaaaatagaagcggccattcaagagctgaactatcagccgaatttcgtcgcccgcagcttaaaacagaaatcaacatttacggtaggtgtggtggtggctaatattcttcacactttttccactcaggtcatcagggcggtggaagactattttcatgaacaaggatttcatattattgtctgcaatgcggatgatgaaacggagaaagaaaagaaatatattgaaatgctgcgggctaagcaggtggacggaattattatttttccgacgggcgaaaatctctctttatatgaaaagatgaaacgcgatacgtttccggttgtgtttatggacagaaccattgaagaactcggaattccgacagtgatgcttgataatcatcatgcagccggtcttgccgttgaccggtttatcgaaagcggcattaagagaatcgcaattattacgacgtctattattcgggaaatcagtccgagggtagaaaggattgaagggtataaaaaggcgcttgaccgtcacggaatgatggtgcgggacgaatatatcaaaacagccgatgcggctgatatttccaatgttttgtcagagctgttctcacttgaggagcctccgaaagcgatattggcggcaaacgatatcgttcttgttgaggtgctgaagtatatgaaggagcatgatatgacaattccggataaggcagccgtcatcggaattgatgaagttccttttgcgggtttttttactcctccgattacgacgatcgtccagcctgcggcagaaatggcgcgaaaagcgggaagccttcttcttcgccagatacaagaaaaagatggcggtgaaaaacggattcaccgctataaaccggcgctgctcgcaaggcaatccggataa

SEQ ID NO: 16 - protein, 333 aa, Bacillus amyloliquefaciens
MKTITISDVAKYANVSKSTVSQFLNQRYDYMSEKTKQKIEAAIQELNYQPNFVARSLKQKSTFTVGVVVANILHTFSTQVIRAVEDYFHEQGFHIIVCNADDETEKEKKYIEMLRAKQVDGIIIFPTGENLSLYEKMKRDTFPVVFMDRTIEELGIPTVMLDNHHAAGLAVDRFIESGIKRIAIITTSIIREISPRVERIEGYKKALDRHGMMVRDEYIKTADAADISNVLSELFSLEEPPKAILAANDIVLVEVLKYMKEHDMTIPDKAAVIGIDEVPFAGFFTPPITTIVQPAAEMARKAGSLLLRQIQEKDGGEKRIHRYKPALLARQSG

SEQ ID NO: 17 - DNA, 489 bp, Bacillus amyloliquefaciens
  source 1..489: mol_type = genomic DNA
atgttctcaaaagatagattccctattattcttcttttattttcagccggggtgattaactatttagaccgctcagcgctgtctattgcagcgccgttcatacagcaggatctcacattgtctgcaacccaaatggggctgattttcagcagtttttctgtcggatacgctgtgtttaactttctcggcggtgttgcatctgaccggtatggggctaaattgacattgtgtacggcgatgatcgtttggtctcttttcagcggagcggttgcgcttgctttcggatttgtaagtcttttgattatccgtgttttattcggtatgggagaggggccgctgtcggctgccataagcaaaatggtcaacaattggtttcctccgtctcagcgcgccacggtcatcggcctgacaaacagcggcacgccgctcggccggttttggattgatgcttggttaattggatggcggagcttggcagtgccgcgatga

SEQ ID NO: 18 - protein, 162 aa, Bacillus amyloliquefaciens
MFSKDRFPIILLLFSAGVINYLDRSALSIAAPFIQQDLTLSATQMGLIFSSFSVGYAVFNFLGGVASDRYGAKLTLCTAMIVWSLFSGAVALAFGFVSLLIIRVLFGMGEGPLSAAISKMVNNWFPPSQRATVIGLTNSGTPLGRFWIDAWLIGWRSLAVPR

SEQ ID NO: 19 - DNA, 1266 bp, Bacillus amyloliquefaciens
  source 1..1266: mol_type = genomic DNA
atgaaaaaaatgttatttatgctggcggtttgtttatgtatgatccctgcggacgtctatgccgctgatctgggacggcagacgttaggaacaaatgacggctggggagccgcttccggcgggacaaccggcggcgcgaaagcctcttcatcgaatgtatacaccgtatcaaacagacagcagctcgtttctgcattagggggaagcgccaacagcacaccgaagattatttacatacaaggcacaatcaatatgaatacggacgaccgcaataaaacgctcggctttgatgattataaagatccggcatatgatccgaacgcctatttgaaggcgtacgacccggacaaatggggtaaaaaagcgccgtcaggcacccaggaagacgccagacagcgatcgcaaaaaaaccagaaggcacgggtcgtggttgacattccttcaaatacgacgatcatcggctcgggttcaaatgccaaagtgacaggcgggaattttaatattaagaacggcgtcgacaatgtcatcatccgcaacattgaatttcaagacgcttacgactattttccgcaatgggacccgacggacggcagcagcggcaactggaactcggaatatgacaatatcaccattaacggcgcgacgcacatctggatcgatcattgcacctttaatgacggatcgaatcccgacagcagttttccctactattacgggagaaaatatcagcaccatgacggccagacggatatagccaacggggcgaattatattacattgtcctataataaatatcatgaccatgacaaaggctccgtcatcggaaacagcgacagcaagacgtcggatgaaggaaaactgaaggtgaccattcaccataactattaccagaacatcgtccagcgcgcaccgcgggtccggtacgggcaggttcatatttataataatttttacgcaggctctaaaagcgccgcatacccgttcagctatgcgtggggcgcgggacacgcgtcaaaaatatatgctcagaataatgtctttgaagtgccgggtctggctgctgataaggtcatcagcgtcttcagcggcggaaaagcgcttcatgaagacggcactcttttaaacggcgccgccattaacgcgtcagccgccaacggattaagtcagtccgtcggctggacaccgtcattgcacggttctatcggctcatcatcaaatgtaaaatcagatgttatatctaaagccggagcgggcatattaaaataa

SEQ ID NO: 20 - protein, 421 aa, Bacillus amyloliquefaciens
MKKMLFMLAVCLCMIPADVYAADLGRQTLGTNDGWGAASGGTTGGAKASSSNVYTVSNRQQLVSALGGSANSTPKIIYIQGTINMNTDDRNKTLGFDDYKDPAYDPNAYLKAYDPDKWGKKAPSGTQEDARQRSQKNQKARVVVDIPSNTTIIGSGSNAKVTGGNFNIKNGVDNVIIRNIEFQDAYDYFPQWDPTDGSSGNWNSEYDNITINGATHIWIDHCTFNDGSNPDSSFPYYYGRKYQHHDGQTDIANGANYITLSYNKYHDHDKGSVIGNSDSKTSDEGKLKVTIHHNYYQNIVQRAPRVRYGQVHIYNNFYAGSKSAAYPFSYAWGAGHASKIYAQNNVFEVPGLAADKVISVFSGGKALHEDGTLLNGAAINASAANGLSQSVGWTPSLHGSIGSSSNVKSDVISKAGAGILK

SEQ ID NO: 21 - DNA, 1407 bp, Bacillus amyloliquefaciens
  source 1..1407: mol_type = genomic DNA
atgaaaataaaattatcgattttatccgctgcggttctggccgcaggcattaccgcctttgtctggccaaaaacagaggccaatgaaaaaacgcagacggacgccgtgtatgtttctcccgcaggcagcgaccaaaatgaaggaacgttagaaaagccgttccgcacattaaaacatgcggctgaaaaggcagaggccggaacaaccgtattgattcgggaagggacgtacgatgaaacgtttgaagtaaagcacagcggcacggccgaaaagccgattacatttcggaactatcagaatgagcatgtctcaatcagcggcaaatctgcgccaaaaagcgattctgaaactccgttaattcaaatccgcaataaacagtacattaccattcacggcgttacgcttgagaacctctccgtatcatcagaagatgcgactgccatggggatctgcgtcaccggatcaagcagtcatatcaatattgagggcaaccatatccgaaacattaaaaccacggctgatgagggaaatgcacacggcatcgcgttttacggcacaggcgccatgaaggatgtcagcatcacaaataatacggttgaaaaactgacgctcggcgcaagcgaagcggtcgtgctgaacgggaatgttgacggatttaagattgccggcaataccattcgggacaataacaatatcgggattgacgtgatcggatacgaaggaacgtccaagcaaaacgactatgcgcgaaacggcgtcattgaaaacaatacggtgagccataactcttcttacggaaatcccgcttacggagatgaatattcagcgggcggtatttacgttgacggcgcagaacatgtggacattaagaagaacaccgtttacaacaatgacctcggcattgaagccacttccgaacatagagggaaatacgcacgagatatccggattacggataacaaggtgtacggcaatgcttacaccggtatttcaatcggaggctatgacacgaaacgcggcggcaccatcaattcggtgatcgctcataacattatgtatcgaaatgacacgaaggaccttgacggcggccagctgctgctgcagtacggcacaaaagggaacacgatcgaaaaaaacatcatgacggcaagcggttcacggatttttatcgcaaatgattatacgaaaaacgaaggcaatactgttaatcataacgtctatcacaaagaagccggaaaagacggcatttggaattggaagaacagagaatacgactcattctctgcttaccgaaaaggaacggcaaacgattcggattccatttatgcggacccgatgtaccgtgacgaatcgtcttatgactttacattaaagcccggatcaccggccctgcccgtcattcagtaa

SEQ ID NO: 22 - protein, 468 aa, Bacillus amyloliquefaciens
MKIKLSILSAAVLAAGITAFVWPKTEANEKTQTDAVYVSPAGSDQNEGTLEKPFRTLKHAAEKAEAGTTVLIREGTYDETFEVKHSGTAEKPITFRNYQNEHVSISGKSAPKSDSETPLIQIRNKQYITIHGVTLENLSVSSEDATAMGICVTGSSSHINIEGNHIRNIKTTADEGNAHGIAFYGTGAMKDVSITNNTVEKLTLGASEAVVLNGNVDGFKIAGNTIRDNNNIGIDVIGYEGTSKQNDYARNGVIENNTVSHNSSYGNPAYGDEYSAGGIYVDGAEHVDIKKNTVYNNDLGIEATSEHRGKYARDIRITDNKVYGNAYTGISIGGYDTKRGGTINSVIAHNIMYRNDTKDLDGGQLLLQYGTKGNTIEKNIMTASGSRIFIANDYTKNEGNTVNHNVYHKEAGKDGIWNWKNREYDSFSAYRKGTANDSDSIYADPMYRDESSYDFTLKPGSPALPVIQ

SEQ ID NO: 23 - DNA, 1065 bp, Bacillus amyloliquefaciens
  source 1..1065: mol_type = genomic DNA
atgataaagaagacgcgtcatctcgctttcttggctgcactcggctttgcgctttgtctggccatcgtatgttccgcatcaaagcaggcagaagcggcagcggcttatccggatgtgatgcagggacttaccggattcgcgggaaatgcgaaggatcataacggaaaagcaaaatccgccgtaacgggaggccagggcggccccgtcgtctacgtcagcaatctcaatgatttgaaaaacaatgcgggcggaacggaccgcaaaaccatcgtcatcacaagtgatatttcttccccgggtaaagccgtcgtgacagtcggcgccaataaaaccatcgtcggctcttataccagccacaggctgaccaacatctatctgaccaccggctccggctcaaacaatgtgattttcaaaaatctcattatcagccatagtgccgccattacgggcaacaacgacattccgatgtacatagcgaacggacagaattactggattgaccacgtgtggtttgaaggacacagctacaatccgaacagccacagtgatttgggaaagcttctgtatgtcggcgccaaagcggattttgtgacgctgtctaactctaaattcacagaccatctgtacgggctgattctcggttatccgaatgatgataacgaaggacgcaattatatcggctatccgcatatgacgatcaccaacaactattttaataacgtctatgtgcgttcaccgggcctgatgagatacggctattttcacgcgaaaaacaactatgtgaccaatttcaatttaggattcaccattcatacaaacgcgactgtcttctcagaagccaactatttcggtaacggaaatgaaaaaggcggaatgatagacgactacgggacggcccaattcaccgacaccggttcattcccgtcgctcaaggccccgaaatcaccgcgcaccggctggaatccgagatcaaattacagctacggcaccctttcagcccaggacgccaaaaacttcgcccaatcttacgcgggagcgcaaaatacaaatctccgctatccataa

SEQ ID NO: 24 - protein, 354 aa, Bacillus amyloliquefaciens
MIKKTRHLAFLAALGFALCLAIVCSASKQAEAAAAYPDVMQGLTGFAGNAKDHNGKAKSAVTGGQGGPVVYVSNLNDLKNNAGGTDRKTIVITSDISSPGKAVVTVGANKTIVGSYTSHRLTNIYLTTGSGSNNVIFKNLIISHSAAITGNNDIPMYIANGQNYWIDHVWFEGHSYNPNSHSDLGKLLYVGAKADFVTLSNSKFTDHLYGLILGYPNDDNEGRNYIGYPHMTITNNYFNNVYVRSPGLMRYGYFHAKNNYVTNFNLGFTIHTNATVFSEANYFGNGNEKGGMIDDYGTAQFTDTGSFPSLKAPKSPRTGWNPRSNYSYGTLSAQDAKNFAQSYAGAQNTNLRYP

SEQ ID NO: 25 - DNA, 1577 bp, Bacillus amyloliquefaciens
  source 1..1577: mol_type = genomic DNA
gatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtctgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagagccgcgggtaatacgtaggtggcaagcgttgtccggaattattgggcgtaaagggctcgcaagcgttttcttaagtctgatgtgaaacccccgggctcaaccggggagggtcattggaaaccgaggaacttgagtgcagaagaggagagtggaattccacgtgtagcggtgaaatgcgtagagatgtggaggaacaccagtggcgaaggcgactctctgttctgtaactgacgctgagagagcgaagcgtggggagcgaacagaattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttatggagccagccgccgaaggtgggacagatgattggggtgaagtcgtaacaaggtagccgtatcggaaggtgcggctggatcacctcctttctaaggattttaacggaatataagaccttgggtcttataac

SEQ ID NO: 26 - DNA, 1836 bp, Bacillus amyloliquefaciens
  source 1..1836: mol_type = genomic DNA
atgtacatcggatcaactaacagcaaaggccttcaccacttggtgtgggaaatcgtcgacaacagtattgacgaagccctggccggttattgtacagatattaacatcgagattgaaaaagataacagcattaccgttaaggacaacgggcgcggaattccggtcggtatccaggagaagatgggccgccctgcggttgaagtcatcatgaccgttctccacgccggcggtaaatttgacggaagcggatataaagtatccggcggtcttcacggtgtaggggcatctgtcgtaaacgccttgtcgaccactcttgacgttacggttcatcgtgacggaaaaatccactatcaggcgtacgagcgcggtgtacctgtggccgatcttgaagtgatcggtgatactgataagaccggaacgattacgcacttcgttccggatccggaaatcttcaaagaaacaatcgtatacgactatgatctgctttcaaaccgtgtccgggaattggccttcctgacaaaaggcgtaaacatcacgattgaagacaaacgtgatggacaagaacggaaaaacgagtaccactacgaaggcggaatcaaaagctatgttgagtacttaaaccgttccaaagaagtcgttcatgaagagccgatttatatcgaaggcgagaaagacggcataacggttgaagttgcattgcaatacaacgacagctatacaagcaacatttattctttcacaaataatatcaacacttacgaaggcgggacgcacgaagccggatttaaaaccggtctgacccgtgtcataaacgactatgcaagaagaaaagggattttcaaagaaaatgatccgaacttaagcggggatgatgtgagagaagggctgactgccattatttcaattaagcaccctgatccgcaattcgaagggcagacgaaaaccaagctcggcaactccgaagcgagaacgatcactgatacgctgttttcttctgcgctggaaacattccttcttgaaaatccggactcagcccgcaaaattgttgaaaaaggtttaatggccgcaagagcgcggatggcagcgaaaaaagcacgggaattgacccggcgcaaaagtgcgcttgagatttccaatctgccgggcaaactggcggactgttcttctaaagatccgagcatttccgagctgtatatcgtagagggtgactctgcgggcggatcagcgaaacagggacgggaccgtcatttccaagctattctgccgctgcgcggtaagattctgaacgttgagaaagccagacttgataagattctctcaaacaatgaggtcagatcaatgatcacggccctcggaacaggaatcggagaagattttaatcttgaaaaagcccgttatcataaagtggtcatcatgacggatgccgatgttgacggcgcccacatcagaacgcttttattaacgttcttctacagatacatgcgggaaatcatcgaaaacggctatgtctacattgcccagccgccgctttataaagtgcagcagggaaaacgggtggaatacgcttataacgataagcagcttgatgagctgttaaaagaacttccgcaatcacctaagcccggcctccagcgttataaaggtcttggagaaatgaacgcgactcagctttgggaaacgacaatggaccctgcgaccagaacgcttctgcaagtcaatcttgaagatgcaatggacgctgacgagacttttgaaatgctgatgggtgacaaagtagaaccgcggagaaacttcatagaagcaaacgccagatacgtgaaaaaccttgatatttaa